(19) United States
(12) Patent Application Publication
(10) Pub. No.: US 2013/0024123 A1
(43) Pub. Date: Jan. 24, 2013

(54) METHODS AND SYSTEMS FOR DETERMINING PHYSIOLOGICAL PARAMETERS USING TEMPLATE MATCHING

(71) Applicants: James Ochs, Seattle, WA (US); Paul Addison, Edinburgh (GB); James Watson, Dunfermline (GB)

(72) Inventors: James Ochs, Seattle, WA (US); Paul Addison, Edinburgh (GB); James Watson, Dunfermline (GB)

(73) Assignee: Nellcor Puritan Bennett Ireland, Mervue (IE)

(21) Appl. No.: 13/188,334

(22) Filed: Jul. 21, 2011

(51) Int. Cl. G06F 19/10; A61B 5/00

(52) U.S. Cl. 702/19; 600/300

(57) ABSTRACT

A patient monitoring system may be configured to use template matching in determining physiological parameters. A physiological signal may be monitored, and a wavelet transform may be performed. The wavelet transform, or parameters derived thereof such as energy distribution or relative phase difference, may be compared with one or more templates using template matching. Templates may be based on, for example, physiological data, mathematical models, or look-up tables, and may be pre-computed and stored. Physiological parameters may be determined based on the template matching results. Scale variability, confidence metrics, or both, may be used to aid in determining the physiological parameter.

The present disclosure relates to template matching, and more particularly relates to comparing wavelet transforms to templates to determine physiological parameters.

### SUMMARY

A patient monitoring system may be configured to determine a physiological parameter using one or more templates. The system may calculate a wavelet transform for a physiological signal. The wavelet transform, or parameters derived thereof, may be compared with each of the one or more templates, and a particular comparison may be selected. Comparisons may include correlation calculations (e.g., a covariance value), difference calculations, any other suitable comparison, or any combination thereof. Templates may include an expected energy distribution across scale, relative phase difference across scale, any other suitable information, or any combination thereof. A physiological parameter may be determined based at least in part on the selected comparison. In some embodiments, a determined physiological parameter may be filtered to limit value, rate of change, or other behavior.

In some embodiments, a scale variability signal may be determined based at least in part on a calculated wavelet transform, or metric derived thereof. For example, a combined signal may be calculated based at least in part on the plurality of comparisons and the scale variability signal. A combined signal may be calculated by multiplying at each scale, multiplying at each scale using a weighted product, summing at each scale, summing at each scale using a weighted sum, any other suitable calculation, or any combination thereof. In some embodiments, a combined signal may be time or ensemble averaged.

In some embodiments, calculating a wavelet transform for template matching may include using a wavelet whose oscillatory character (e.g., a characteristic frequency) depends on a scale such as, for example, stepwise decreasing as scale increases, linearly decreasing as scale increases, nonlinearly decreasing as scale increases, any other suitable scale dependence, or any combination thereof.

In some embodiments, a confidence metric may be calculated and used to aid in averaging, filtering, or combining calculated signals or parameters.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a patient, in accordance with some embodiments of the present disclosure;

FIG. 3(a) shows an illustrative view of a scalogram derived from a photoplethysmograph (PPG) signal, in accordance with some embodiments of the present disclosure;

FIG. 3(b) shows an illustrative view of the scalogram of FIG. 3(a), in accordance with some embodiments of the present disclosure;

FIG. 3(c) shows an illustrative scalogram derived from a signal containing two pertinent components, in accordance with some embodiments of the present disclosure;

FIG. 3(d) shows an illustrative schematic of signals associated with a ridge of FIG. 3(c) and illustrative schematics of a further wavelet decomposition of these newly derived signals, in accordance with some embodiments of the present disclosure;

FIG. 3(e) is a flowchart of illustrative steps involved in performing an inverse continuous wavelet transform, in accordance with some embodiments of the present disclosure;

FIG. 3(f) is a flowchart of illustrative steps involved in performing an inverse continuous wavelet transform, in accordance with some embodiments of the present disclosure;

FIG. 4 is a block diagram of an illustrative continuous wavelet processing system, in accordance with some embodiments of the present disclosure;

FIG. 5 shows an illustrative energy distribution of a continuous wavelet transform across scales using a fixed f₀, in accordance with some embodiments of the present disclosure;

FIG. 6 shows an illustrative energy distribution of a continuous wavelet transform across scales using a variable f₀, in accordance with some embodiments of the present disclosure;

FIG. 7 shows an illustrative energy distribution template for 60 BPM, in accordance with some embodiments of the present disclosure;

FIG. 8 shows an illustrative energy distribution template for 150 BPM, in accordance with some embodiments of the present disclosure;

FIG. 9 shows an illustrative energy distribution template for 300 BPM, in accordance with some embodiments of the present disclosure;

FIG. 10 shows an illustrative algorithm for comparing with one or more templates, in accordance with some embodiments of the present disclosure;

FIG. 11 is an illustrative diagram of the algorithm of FIG. 10, in accordance with some embodiments of the present disclosure;

FIG. 12 shows an illustrative time series of an infrared (IR) photoplethysmograph (PPG) derivative, in accordance with some embodiments of the present disclosure;

FIG. 13 shows an illustrative energy distribution of the continuous wavelet transform of the signal of FIG. 12, exhibiting an artifact, in accordance with some embodiments of the present disclosure;

FIG. 14 shows an illustrative inverse measure of scale variability, in accordance with some embodiments of the present disclosure;

FIG. 15 is a diagram showing the combination of template matching results with scale variability to create a combined signal, in accordance with some embodiments of the present disclosure;

FIG. 16 is a flow diagram showing illustrative steps for determining a physiological parameter, in accordance with some embodiments of the present disclosure;

FIG. 17 is a flow diagram showing illustrative steps for using template matching and scale variability to create a combined signal, in accordance with some embodiments of the present disclosure; and

FIG. 18 is a flow diagram showing illustrative steps for determining a physiological parameter using template matching, scale variability, and confidence metrics, in accordance with some embodiments of the present disclosure.

### DETAILED DESCRIPTION OF THE FIGURES

The present disclosure is directed towards using template matching to determine physiological parameters. A patient monitoring system may monitor one or more physiological parameters of a patient, typically using one or more physiological sensors. The patient monitoring system may condition signals received from the sensor, perform suitable mathematical calculations on the conditioned signals, and extract values of a physiological parameter. For example, a patient monitoring system may perform a wavelet transform on a received and conditioned (e.g., amplified, filtered, sampled, digitized, etc.) photoplethysmograph signal (or mathematically derived signal thereof). The wavelet transform provides both time and scale information of the conditioned signal. Further calculations may be performed on the wavelet transform including the energy distribution across scale, the relative phase difference across scale, any other suitable derived metric across scale, at a particular time or time interval, or any combination thereof. Determination of physiological parameters may be improved by using pre-computed templates which contain information about expected or historical signal behavior. Templates may include averaged signals (e.g., from sample populations, historical data from a particular patient), mathematical models, look up tables, any other suitable pre-computed form, or any combination thereof. In some embodiments, a calculated signal may be compared with a template to aid in determining a physiological parameter. In some embodiments, signal variation (i.e., a measure of expected signal variation across scale) may be used to aid in determining a physiological parameter. For example, a template may include an expected energy distribution across scale while scale variability may include an observed variability in energy across scale over time.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I₀(λ)exp(−(sβ₀(λ)+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I₀−(sβ₀+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{O} ( {λ}_{R} ) + ( 1 - s ) {β}_{r} ( {λ}_{R} )}{s {β}_{O} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{O} ( {λ}_{IR} ) - {β}_{r} ( {λ}_{IR} ) ) - \\ \frac{d log I ( {λ}_{IR} )}{d t} ( {β}_{O} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{\frac{d I}{d t}}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {T}_{1} , {λ}_{IR} )}{[ I ( {t}_{2} , {λ}_{IR} ) - I ( {t}_{1} , {λ}_{IR} ) ] I ( {T}_{1} , {λ}_{R} )} = R , \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

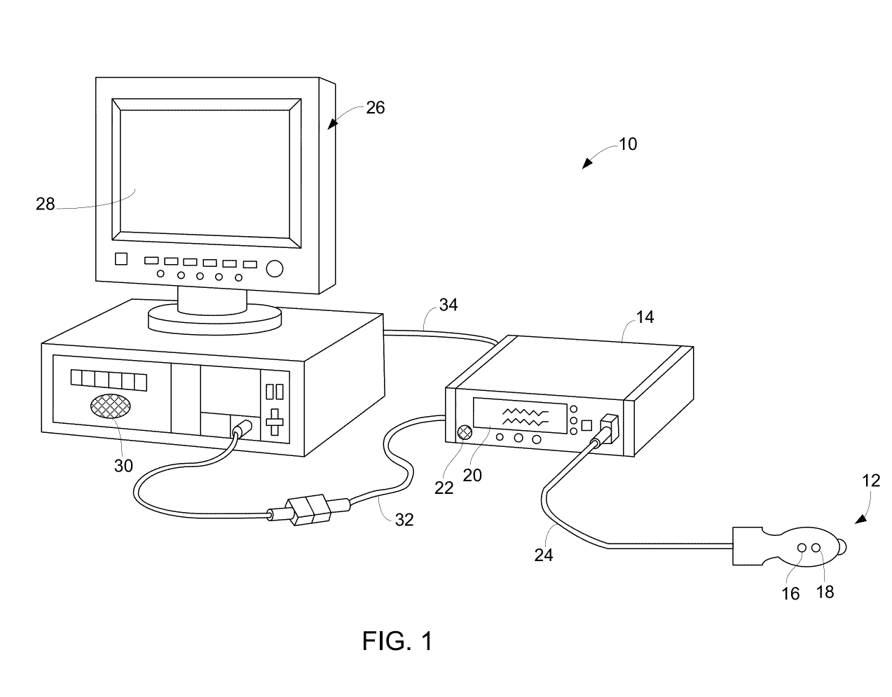

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) which may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include a plurality of sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In an embodiment, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood pressure, blood oxygen saturation) based at least in part on data relating to light emission and detection received from one or more sensor units such as sensor unit 12 and an additional sensor. In an alternative embodiment, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the monitor 14 includes a blood pressure monitor. In some embodiments, the system 10 includes a stand-alone blood pressure monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24.

In the illustrated embodiment, system 10 includes a multi-parameter patient monitor 26. The monitor 26 may include a cathode ray tube display, a flat panel display (as shown) such as a liquid crystal display (LCD) or a plasma display, or may include any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as an “SpO₂” measurement), pulse rate information from monitor 14 and blood pressure from monitor 14 on display 28. Multi-parameter patient monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

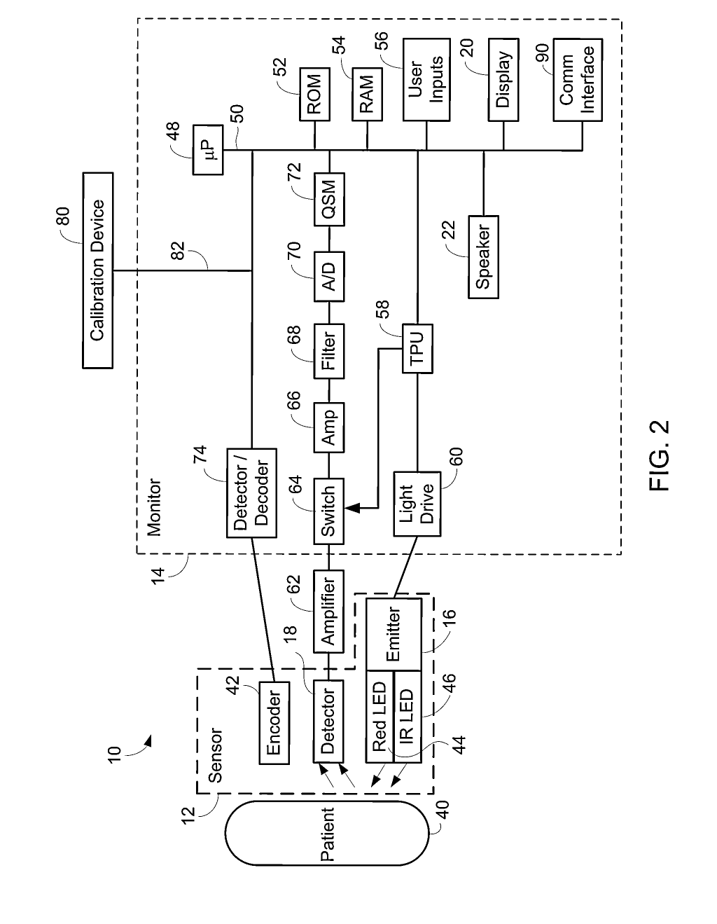

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In one embodiment, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor emits only a Red light while a second emits only an IR light. In another example, the wavelengths of light used are selected based on the specific location of the sensor.

It will be understood that, as used herein, the term “light” may refer to energy produced by radiation sources and may include one or more of ultrasound, radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a portion of a photoplethysmograph (PPG) signal corresponding to a physiological pulse to determine blood pressure. These equations may contain coefficients that depend upon a patient's physiological characteristics as stored in encoder 42. Encoder 42 may, for instance, be a coded resistor which stores values corresponding to the type of sensor unit 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 fills up. In one embodiment, there may be multiple separate parallel paths having components equivalent to amplifier 66, filter 68, and/or A/D converter 70 for multiple light wavelengths or spectra received.

In an embodiment, microprocessor 48 may determine the patient's physiological parameters, such as SpO₂, pulse rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Communications (“Comm”) interface 90 may include any suitable hardware, software, or both, which may allow patient monitoring system 10 to communicate with electronic circuitry, a device, or a network, or any combinations thereof. Communications interface 90 may include one or more receivers, transmitters, transceivers, antennas, plug-in connectors, ports, communications buses, communications protocols, device identification protocols, any other suitable hardware or software, or any combination thereof. Communications interface 90 may be configured to allow wired communication (e.g., using USB, RS-232 or other standards), wireless communication (e.g., using WiFi, IR, WiMax, BLUETOOTH, UWB, or other standards), or both. For example, communications interface 90 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In a further example, communications interface 90 may be configured to access a database server, which may contain a template database. In some embodiments, communications interface 90 may include an internal bus such as, for example, one or more slots for insertion of expansion cards.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals or otherwise identify noise components in order to prevent them from affecting measurements of physiological parameters derived from the sensor signals.

It will be understood that the present disclosure is applicable to any suitable signal and that PPG signals are used merely for illustrative purposes. Those skilled in the art will recognize that the present disclosure has wide applicability to other signals including, but not limited to, other biosignals (e.g., electrocardiograms, electroencephalograms, electrogastrograms, electromyograms, pulse rate signals, pathological signals, ultrasound signals, any other suitable biosignals), dynamic signals, non-destructive testing signals, condition monitoring signals, fluid dynamic signals, geophysical signals, astronomical signals, electrical signals, financial signals, sound and speech signals, chemical signals (e.g., arising from chemical kinetics), meteorological signals (e.g., climate signals), any other suitable signals, or any combination thereof.

In some embodiments, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{∞} x ( t ) ψ * ( \frac{t - b}{a} ) d t \qquad ( 14 )$$

where ψ*(t) is the complex conjugate of the wavelet function ψ(t), a is the dilation parameter of the wavelet and b is the location parameter of the wavelet. The transform given by equation (14) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (e.g., in time) and scale.

The energy density function of the wavelet transform, (i.e., the scalogram), is defined as

S_{R}(a,b)=|T(a,b)|² (15)

where ‘| |’ is the modulus operator. The scalogram may be rescaled for useful purposes. One common rescaling is defined as

$${S}_{R} ( a , b ) = \frac{{\vert T ( a , b ) \vert}^{2}}{a} \qquad ( 16 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unscaled wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram.”

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 17 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{²}^{/2})e^{-t}^{²}^{/2} (18)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{\frac{1}{4}}} {e}^{i2π {f}_{o} t} {e}^{- {t}^{2} / 2} \qquad ( 19 )$$

The Morlet wavelet is a complex sinusoid within a Gaussian envelope where the central frequency f₀ in effect determines the number of significant oscillations of the complex sinusoid within the Gaussian envelope. The oscillatory character of the Morlet wavelet, or any other suitable wavelet, may be parameterized by using such a parameter which may or may not be a proper “frequency” (e.g., oscillatory character may be found in other types of functions such as polynomials, Haar wavelets, Mexican hat wavelets). The term “oscillatory character” describes the number of oscillations of significant amplitude which occur over a particular time (e.g., suitable portions such as one “time constant” of the Gaussian envelope). For example, wavelets having higher oscillatory character may exhibit relatively more oscillations, and may be associated with higher-energy activity as compared to wavelets having less oscillatory character.

While two definitions of the Morlet wavelet are included herein, the function of equation (19) is not strictly a wavelet as it has a non-zero mean (i.e., the zero frequency term of its corresponding energy spectrum is non-zero). However, it will be recognized by those skilled in the art that equation (19) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

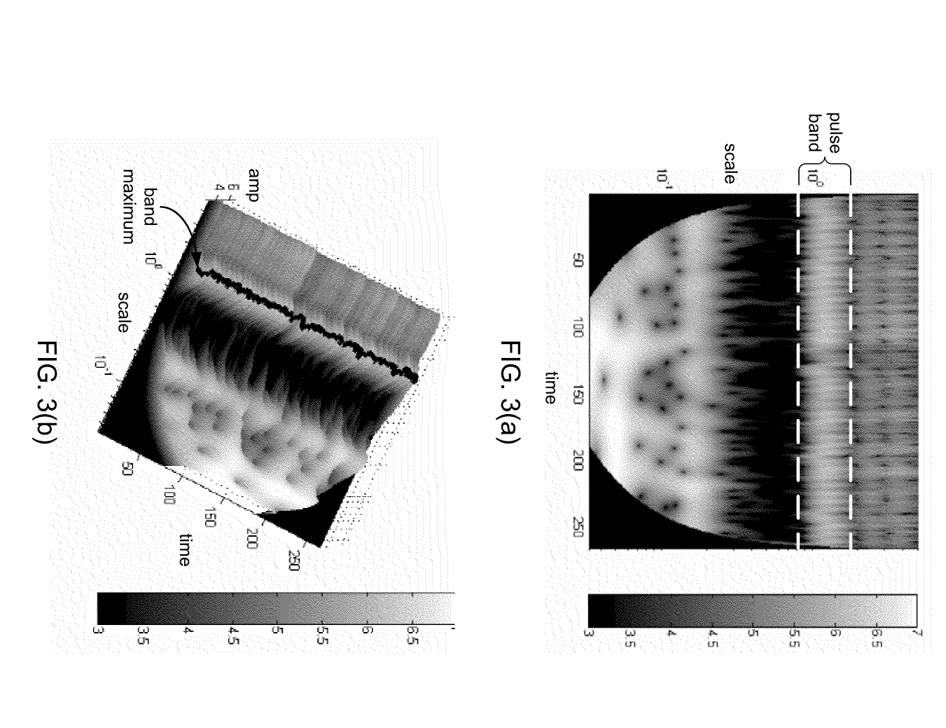

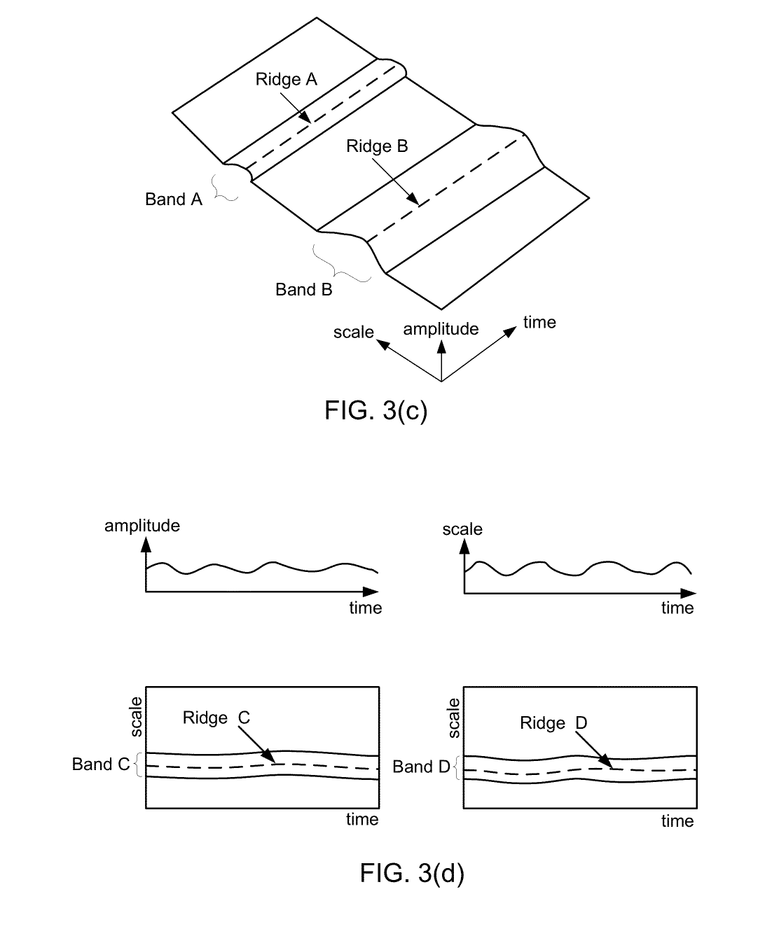

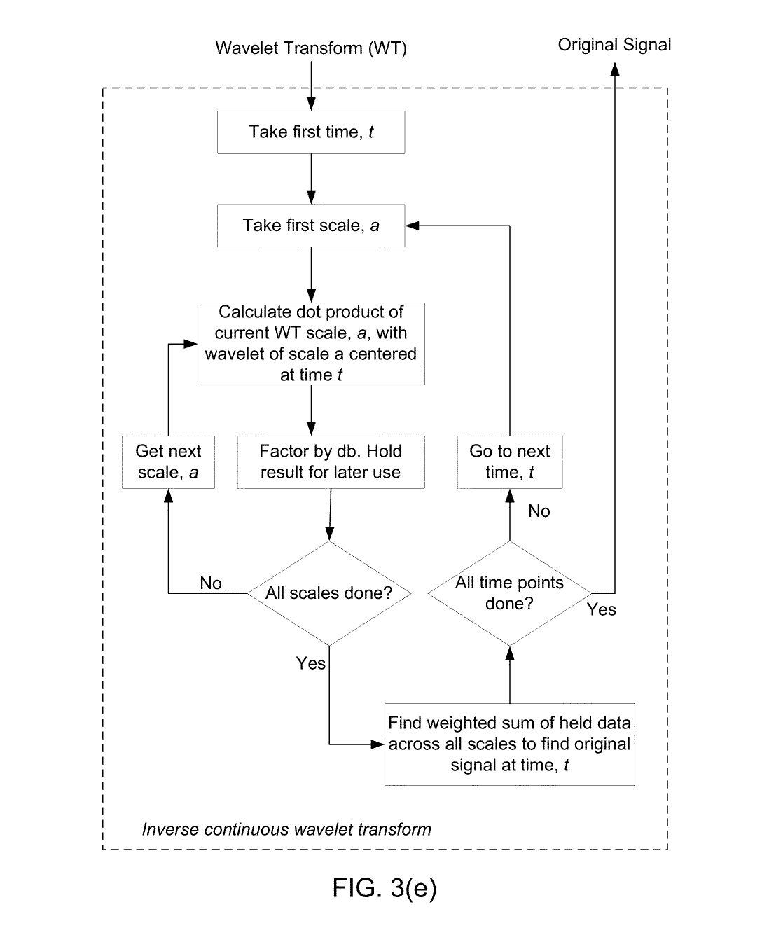

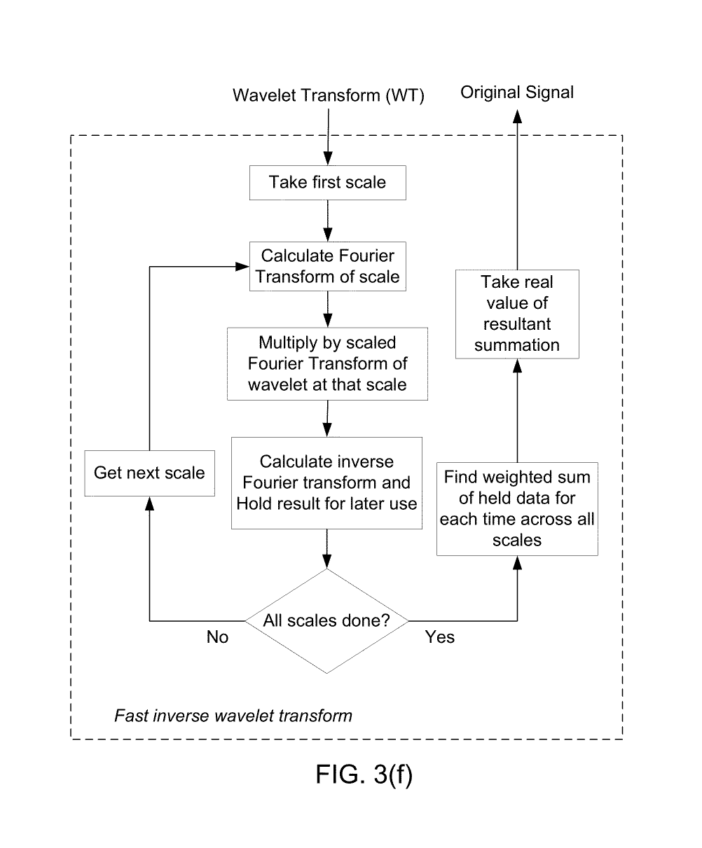

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and 3(b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band, with respect to scale, is the ridge. The locus of the ridge is shown as a black curve on top of the band in FIG. 3(b). By employing a suitable rescaling of the scalogram, such as that given in equation (16), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 20 )$$

which may also be written as:

$$x ( t ) = \frac{1}{{C}_{g}} {\int }_{- ∞}^{∞} {\int }_{0}^{∞} T ( a , b ) \frac{1}{\sqrt{a}} {ψ}_{a , b} ( t ) \frac{d a d b}{{a}^{2}} \qquad ( 21 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 22 )$$

FIG. 3(e) is a flow chart of illustrative steps that may be taken to perform an inverse continuous wavelet transform in accordance with the above discussion. An approximation to the inverse transform may be made by considering equation (22) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

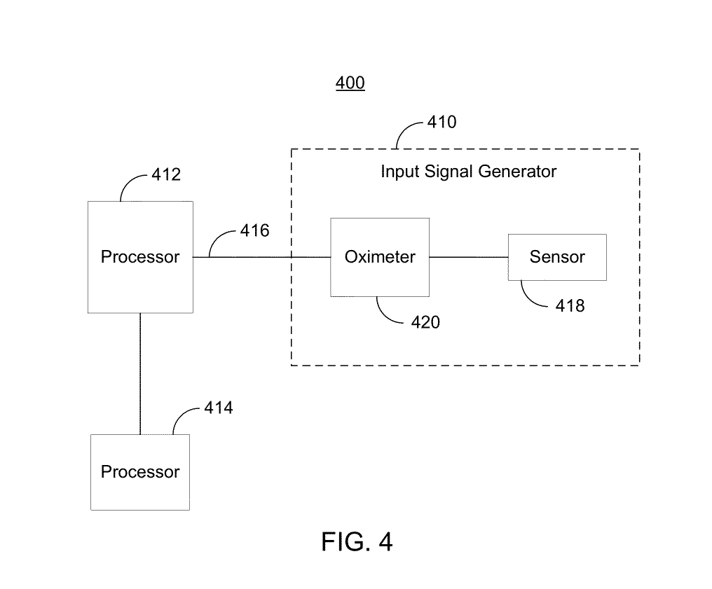

FIG. 4 is an illustrative continuous wavelet processing system in accordance with an embodiment. In this embodiment input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In some embodiments, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

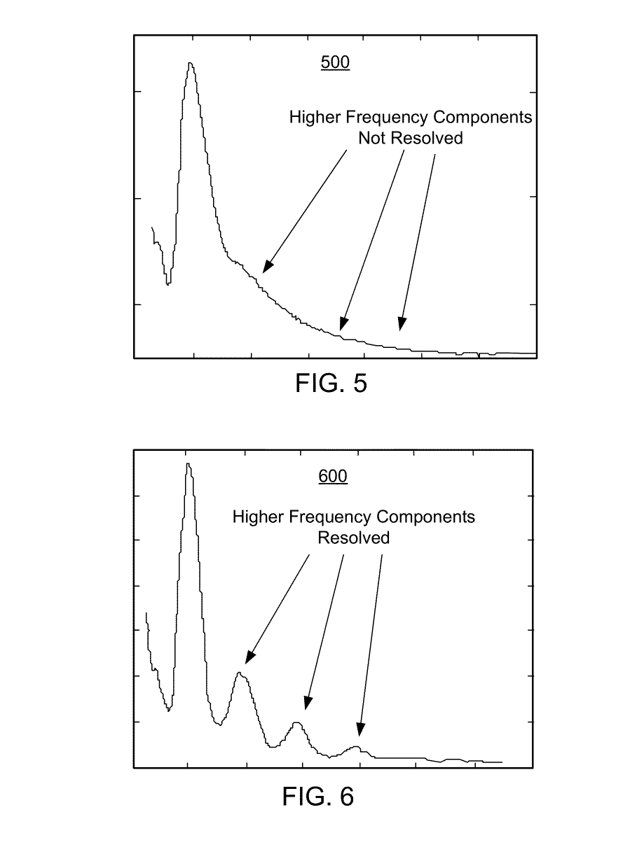

A system, such as patient monitoring system 10 of FIG. 1, may be configured to calculate a continuous wavelet transform, and values derived thereof (e.g., energy distribution, phase difference), for a physiological signal received from a sensor. FIG. 5 shows an illustrative energy distribution 500 of a continuous wavelet transform across scales using a fixed f₀, in accordance with some embodiments of the present disclosure. FIG. 6 shows an illustrative energy distribution 600 of a continuous wavelet transform across scales using a variable f₀, using the same time domain data as that for energy distribution 500 of FIG. 5, in accordance with some embodiments of the present disclosure. The abscissa of FIGS. 5 and 6 are proportional to inverse scale, which may correspond to BPM, while the ordinates are proportional to energy density (e.g., as shown by S(a,b) of equation (15)). Although not shown in FIGS. 5 and 6, each tick mark of the abscissa may correspond to 100 BPM, ranging from 0 to 700 BPM, although the continuous wavelet transform was calculated only for scales corresponding to 20 to 700 BPM. Energy distribution 500 was calculated using an f₀ value of 4.0, while energy distribution 600 was calculated using an that increases by 1.5 for each 30 BPM increase (i.e., 4.0 for 30-60 BPM, 5.5 from 60-90 BPM, etc.)

As shown in FIGS. 5 and 6, use of the variable h may allow higher frequency components to be resolved relative to the use of the fixed f₀. Energy distribution 500 can be seen to peak and then tail off to substantially zero as BPM increases. Energy distribution 600 exhibits a peak similar to energy distribution 500 at lower BPM, but also exhibits several peaks at higher BPM values not resolved by energy distribution 500. For a given scale, increasing the size of variable f₀ generally increases the size (e.g., the number of samples) of the underlying signal that should be used to perform a continuous wavelet transform. In addition, for a given f₀, increasing the scale (i.e., decreasing the characteristic frequency of the wavelet) also generally increases the size of the underlying signal that should be used to perform a continuous wavelet transform. Accordingly, it is possible to increase f₀ with increasing BPM (e.g., corresponding to lower scale) to resolve high-BPM components, but not require long input signals at lower BPMs.

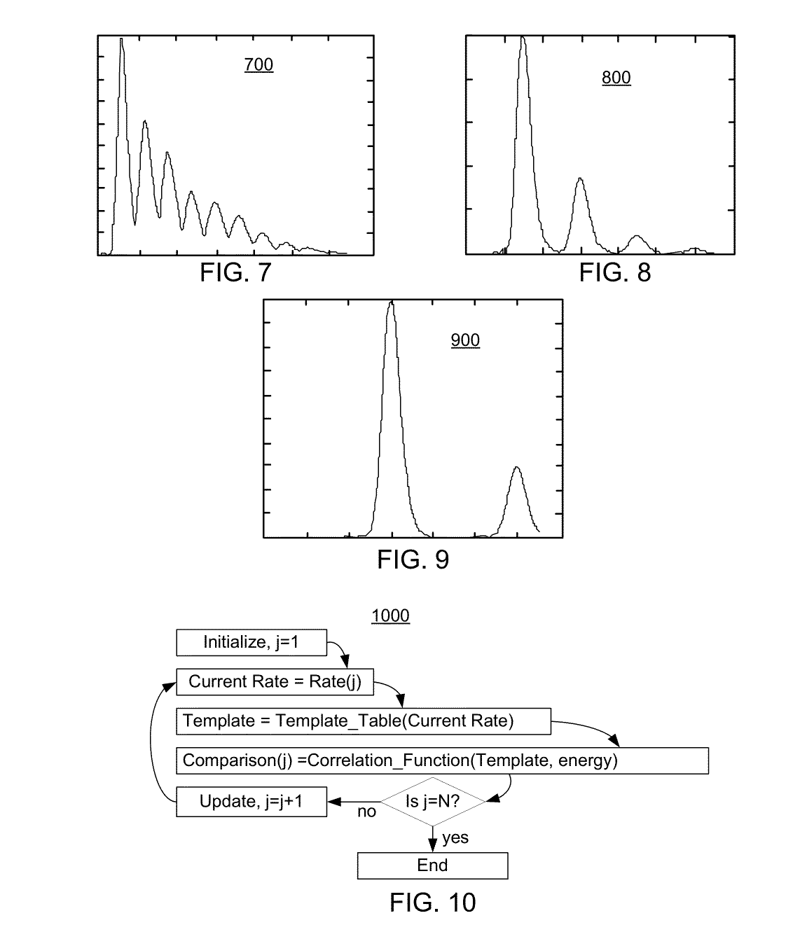

In some embodiments, energy distribution templates may be used for comparison with an energy distribution of a continuous wavelet transform of a physiological signal to determine a physiological parameter (e.g., pulse rate in BPM). Shown in FIGS. 7-9 are illustrative energy distribution templates, corresponding to the “expected” energy distribution for various pulse rates. FIG. 7 shows an illustrative energy distribution template 700 for 60 BPM, in accordance with some embodiments of the present disclosure. FIG. 8 shows an illustrative energy distribution template 800 for 150 BPM, in accordance with some embodiments of the present disclosure. FIG. 9 shows an illustrative energy distribution template 900 for 300 BPM, in accordance with some embodiments of the present disclosure. The abscissa of FIGS. 7-9 are proportional to inverse scale, while the ordinates are proportional to energy density (e.g., as shown by S(a,b) of equation (15)), although normalized by maximum peak height. Although numerical indicators are not shown in FIGS. 7-9, each tick mark of the abscissa may correspond to 100 BPM, ranging from 0 to 700 BPM.

The shape of a PPG signal may change with pulse rate. For example, at lower pulse rates, the PPG signal may exhibit a relatively higher skewness (e.g., the third moment of a pulse wave). At higher pulse rates, the PPG signal may exhibit a more sinusoidal (e.g., non-skewed) character. Lower pulse rates may tend to have multiple significant components corresponding to frequencies above the fundamental pulse rate. Higher pulse rates may tend to have a majority of the pulse energy at a single scale. Templates taking into account the variation in PPG shape with pulse rate may aid in determining the pulse rate from PPG signals. Energy distribution templates 700-900 of respective FIGS. 7-9 illustrate aspects of this variation with pulse rate.

Templates may be generated based on calculated wavelet transform parameters (e.g., wavelet transform values, energy distribution values, or relative phase difference values). In some embodiments, templates may be generated by a patient monitoring system by averaging energy distributions derived from wavelet transforms performed on physiological signals. In some embodiments, time averaging or ensemble averaging may be used to generate templates. For example, templates may be generated from averaged noise-free, PPG signals from one or more patients. In some embodiments, energy distribution templates may be normalized (e.g., such that the maximum energy is 1, or such that the integral of energy over scale is 1). In some embodiments, relative phase differences may be averaged, normalized, or otherwise processed to generate templates in addition to, or in lieu of, energy distributions. In some embodiments, templates may include mathematical models or look-up tables. For example, a template may include a polynomial function of scale (e.g., a least squares polynomial fit to sample data). In a further example, a template may include a polynomial (or any other suitable mathematical representation) with one or more adjustable parameters which may be optimized based on a patient's PPG characteristics (e.g., the presence of a dichrotic notch). Any suitable metric may be calculated by a patient monitoring system and be stored as a template. Templates may be pre-computed (e.g., prior to comparison with wavelet transform parameters) and stored in memory (e.g., a database of templates). The memory may be included in the patient monitoring system (e.g., ROM 52 of patient monitoring system 10), or the memory may be located remotely from the patient monitoring system (e.g., accessible by communications interface 90 of patient monitoring system 10). In some embodiments, templates may be generated remotely (e.g., by a remote processor running a template generating application) and communicated to the patient monitoring system via a suitable communications interface (e.g., communications interface 90 of FIG. 2). Templates may be generated and stored by any suitable combination of processing equipment and memory, in accordance with the present disclosure.

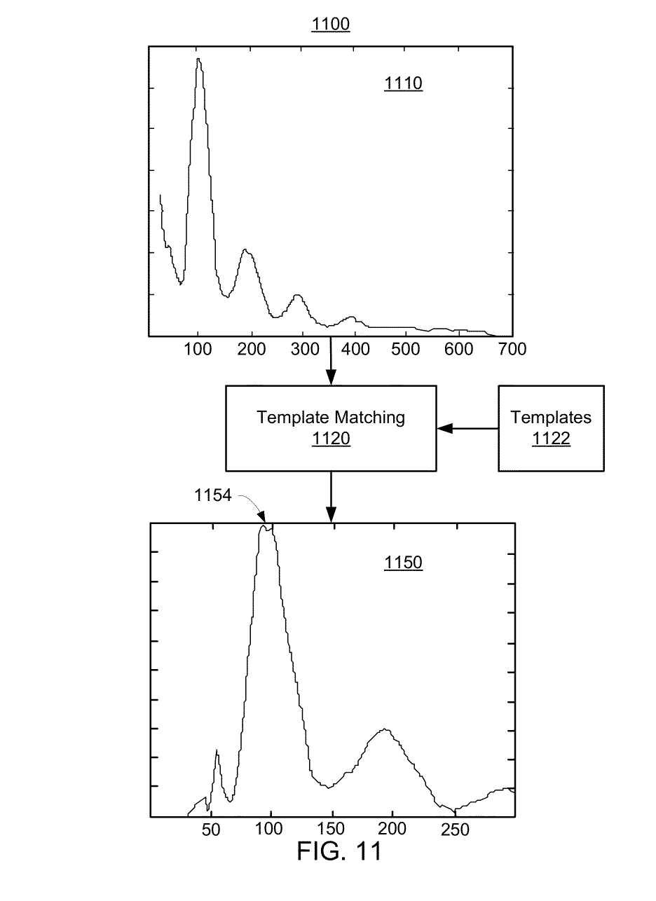

FIG. 10 shows an illustrative algorithm 1000 for comparing a calculated energy distribution with one or more templates corresponding to one or more BPM rates, in accordance with some embodiments of the present disclosure. Algorithm 1000 may be used to compare an energy distribution of a calculated wavelet transform to each of N templates. The number of templates N may be any suitable positive integer (i.e., one or more templates). As one example, there may be one template for each BPM in a typical physiological range of heart rates (e.g., from 20-300 BPM). In the illustrated example, and index j is used to sequentially perform comparisons between the energy density of the calculated wavelet transform (“energy” in algorithm 1000) and each template.

Referencing FIG. 10, starting with an initial index j=1, a first “Rate” (i.e., BPM) is selected and a first template (“Template” in algorithm 1000) is accessed from the plurality of templates (“Template_Table” in algorithm 1000). A comparison is made, such as a correlation in the illustrated example, for the accessed template. The comparison may include any suitable mathematical calculation for comparison, including calculating an absolute difference, a weighted difference, a sum of differences, any other suitable calculation, or any combination thereof. The process is then repeated a sufficient number of times (e.g., until the index j has advanced to N or until a comparison is sufficiently good) and the comparisons are completed. Although illustrative algorithm 1000 shows a sequential advancement through a collection of templates, the comparison need not be performed in sequential order. Any suitable order or algorithm may be used to perform the comparison. In some embodiments, comparisons may be made with a subset of the templates initially, and further comparisons may be made based on the initial comparison results. For example, an initial comparison may be made for every tenth rate, and further comparison may be made for rates relatively near to the best comparison(s) of the initial comparison.

FIG. 11 is an illustrative diagram 1100 of algorithm 1000 of FIG. 10, in accordance with some embodiments of the present disclosure. Energy distribution 1110 is derived from a calculated continuous wavelet transform of a physiological signal across scales (i.e., corresponding to 20 to 700 BPM in this illustrative example). The patient monitoring system may perform template matching 1120, comparing energy distribution 1110 to one or more energy distribution templates 1122, each having a corresponding frequency (e.g., in BPM). In some embodiments, the comparison may provide a single number (e.g., a covariance, a normalized difference) for the comparison performed between energy distribution 1110 and each template of templates 1122.

Template matching results 1150 shows the comparisons of energy distribution 1110 with a plurality of templates corresponding to a range of BPM rates. The comparison may include calculating a correlation (e.g., a covariance value), a difference or sum thereof across scale, any other suitable comparative calculation, or any combination thereof.

Template matching results 1150 shows a large peak at a template frequency of roughly 100 BPM, thus indicating a relatively high correlation of the energy distribution to this template. In some embodiments, the comparison providing the maximum correlation may be selected as the “match”, and the BPM rate associated with the template of that comparison may be characterized as a calculated pulse rate. In some embodiments, template matching results 1150 may be combined with, or considered in view of, other calculated metrics, signals, or both. For example, the correlation value of the best “match” (i.e., the peak value) may be used as a confidence metric. In a further example, template matching results may be compared (e.g., by calculating a covariance value) with the template corresponding to the best match to provide a confidence metric. Any suitable calculated value may be used as a confidence metric.

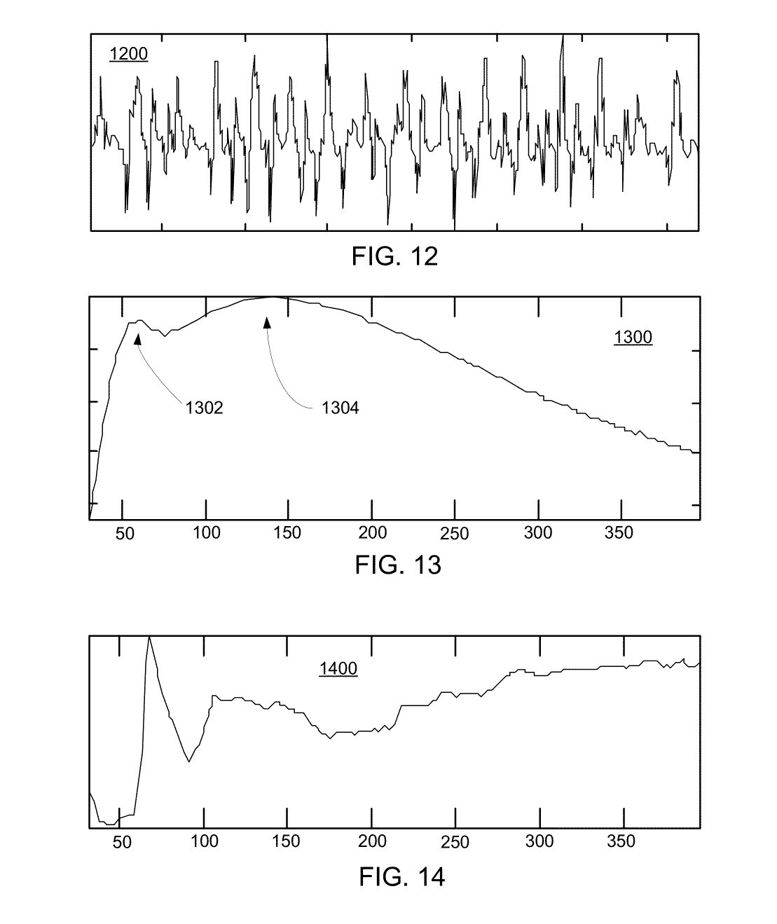

FIG. 12 shows an illustrative time series 1200 of the time derivative of an infrared (IR) photoplethysmograph (PPG), in accordance with some embodiments of the present disclosure. The abscissa of the plot of FIG. 12 is in units of time, while the ordinate is proportional to the derivate of the plethysmograph signal. Time series 1200 may be derived from a photodetector signal, the photodetector detecting IR radiation attenuated by a patient. A patient monitor may receive the photodetector signal, condition the signal (e.g., perform current-voltage conversion, amplify, DC offset, filter, sample, demodulate, convert from analog to digital, and/or any other suitable conditioning steps), and calculate the time derivate of the received signal as shown by time series 1200. It will be understood that a derivative may be, but need not be, performed to perform the disclosed steps and techniques. It will also be understood that the disclosed steps and techniques may be applied to any suitable time series of RED PPG signals, IR PPG signals, or both, any other suitable physiological signal, or any combination thereof.

FIG. 13 shows an illustrative energy distribution 1300 of the continuous wavelet transform (using multiple f₀ values) of time series 1200 of FIG. 12, exhibiting an artifact, in accordance with some embodiments of the present disclosure. The abscissa of the plot of FIG. 13 is in units proportional to inverse scale, while the ordinate is proportional to the energy distribution of the wavelet transform of time series 1200. In the illustrated example of FIG. 13, the true BPM may be given by peak 1302, but artifact 1304 may overlap, interfere with, or otherwise obscure peak 1302. As shown in FIG. 13, selection of the energy distribution maximum (i.e., the peak of artifact 1304) would be misleading in that the maximum does not correspond to the true BPM rate (i.e., peak 1302). In some embodiments, additional calculations or evaluations may aid in ascertaining the “true” pulse rate of a patient, especially when significant artifacts, noise, or both, are present.

FIG. 14 shows an illustrative measure of scale variability 1400, in accordance with some embodiments of the present disclosure. The abscissa of the plot of FIG. 14 is in units proportional to inverse scale, while the ordinate is proportional to scale variability. As shown in FIG. 14, lower values of scale variability indicate less variation over time at a particular scale (or inverse scale). For example, a minimum in scale variability may be observed in FIG. 14 near 50 BPM, indicating that relatively lower variability at the corresponding scale. A scale variability signal may include scale variability values, inverse scale variability values, calculations performed thereon, any other suitable metric describing signal variability, or any combination thereof.

In some embodiments, scale variability may be determined based at least in part on historical physiological signals. For example, a patient's PPG signals may be monitored and the variability, across scale, may be calculated. In some embodiments, scale variability may be determined based at least in part on sample data from a population of patients (e.g., an averaged scale variability calculated based on the sample). For example, scale variability may be determined by calculating the standard deviation of energy at each scale over time, and averaging the standard deviation values. In some embodiments, scale variability may be based at least in part on a mathematical model (e.g., a function of scale, a multi-variable mapping, a conditional probability function), look-up table (e.g., an indexed database), any other mathematical formalism, or any combination thereof.

Scale variability may be calculated at a single scale, multiple scales (e.g., a weighted sum of metrics calculated for various scales), or all scales. For example, wavelet transform parameters for integer multiples of a scale may be added together (e.g., to form a plethysmograph-like signal), and scale variability may be computed for the sum of wavelet transform parameters. Scale variability may be calculated based on the real portion, imaginary portion, or both, of one or more transformed physiological signals (e.g., a wavelet transform) or a signal derived thereof. In some embodiments, after the scales are combined to produce plethysmograph-like signals in wavelet space, scale variability may be calculated based on the variation in both time and amplitude of fiducials of the combined scales (e.g., the standard deviation of fiducials such as peak first derivative, pulse period as determined by the interval between fiducial points of successive pulse waves), variation in shape of the combined scales (e.g., pulse wave area, peak height, skew, kurtosis, dichrotic notch position), any other suitable characteristics of a physiological signal, or any combination thereof. In some embodiments, an inverse wavelet transform may be applied to the combined scales, and one or more fiducials of the resulting time-domain signal may be calculated to provide a measure of variability, confidence, or both.

In some embodiments, a confidence metric may be calculated based at least in part on the scale variability. For example, in some embodiments, a neural network may be trained to calculate a confidence for each scale based on the variability and shape of the real portion of the scalogram at each scale. In a further example, a confidence metric may be based on the extent to which historical or sample population data is available (e.g., higher confidence for larger sample sizes or data collections).

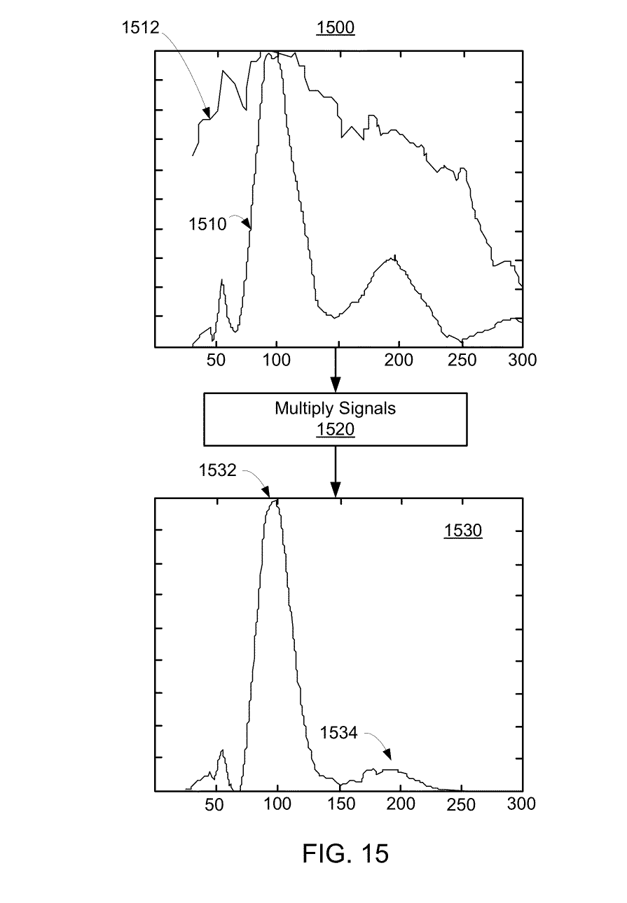

FIG. 15 is a diagram 1500 showing the combination of template matching results 1510 with an inverse measure of scale variability 1512 to create an illustrative combined signal 1530, in accordance with some embodiments of the present disclosure. In the illustrative example shown in diagram 1500, template matching results 1510 and the inverse measure of scale variability 1512 are combined by multiplying the values at each scale, as shown by process 1520. Combined signal 1530, resulting from the multiplication of template matching results 1510 and the inverse measure of scale variability 1512, shows a relatively sharper peak at roughly 100 BPM (i.e., peak 1532) as compared to the primary peak of template matching results 1510. A smaller peak of template matching results 1510 at roughly 200 BPM is observed to be diminished relative to the primary peak (i.e., peak 1532) in combined signal 1530, as shown by peak 1534. The combination of template matching results 1510 with the inverse measure of scale variability 1512 may account for both maximum relative energy and observed variability across scale to provide an improved estimate of the “true” RPM rate.

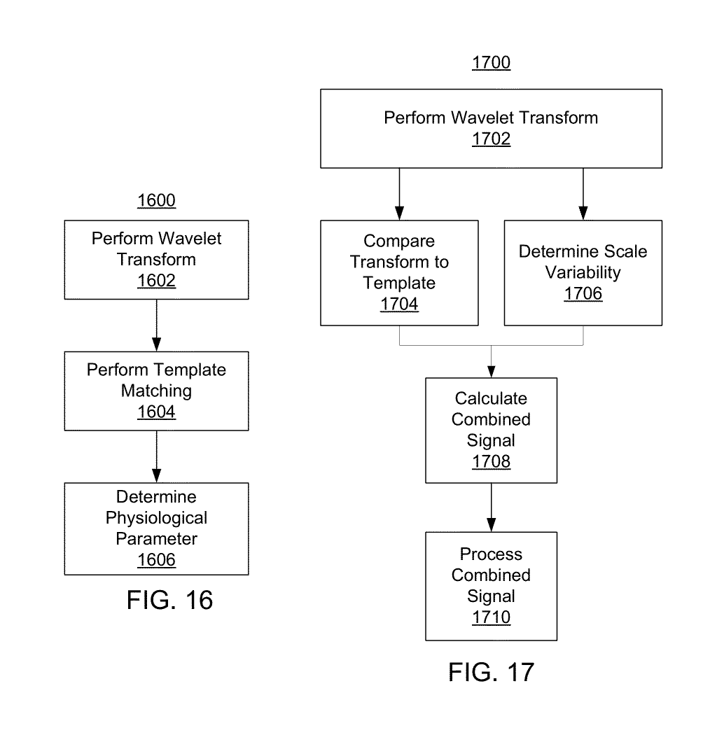

FIG. 16 is a flow diagram 1600 showing illustrative steps for determining a physiological parameter, in accordance with some embodiments of the present disclosure. Any or all of the steps of flow diagram 1600 may be performed by a suitable patient monitoring system (e.g., patient monitoring system 10 of FIGS. 1 and 2), any other suitable system or device, or any combination thereof.

Step 1602 may include a patient monitoring system performing a wavelet transform (e.g., a continuous wavelet transform) on a time-domain physiological signal. In some embodiments, step 1602 may be performed across a range of scales. Step 1602 may output wavelet transform parameters including, for example, the transform values themselves (e.g., T(a,b) of equation (14)), energy distribution values (e.g., S_{R}(a,b) of equation (15)), relative phase difference values, any other suitable derived values, any suitable mathematical manipulations thereof (e.g., scaled S_{R}(a,b) of equation (16)), or any combination thereof. In some embodiments, step 1602 may include generating a scalogram.

Step 1604 may include a patient monitoring system performing a template match between the outputted wavelet transform parameters of step 1602 and one or more templates. Step 1604 may include performing a correlation calculation (e.g., calculating a covariance value for each comparison with each template), performing a difference calculation, performing any other suitable calculation between calculated wavelet transform parameters and one or more templates, or any combination thereof.

Step 1606 may include a patient monitoring system determining a physiological parameter based at least in part on the template match of step 1604. Step 1606 may include selecting a best match from the template matching of step 1604, selecting a template corresponding to the best match, time or ensemble averaging a BPM value associated with the best match template, filtering a calculated BPM rate (e.g., low pass filtering to limit the rate of change in BPM value), any other suitable steps for determining a physiological parameter, or any combination thereof.

FIG. 17 is a flow diagram 1700 showing illustrative steps for using template matching and scale variability to create a combined signal, in accordance with some embodiments of the present disclosure. Any or all of the steps of flow diagram 1700 may be performed by a suitable patient monitoring system (e.g., patient monitoring system 10 of FIGS. 1 and 2), any other suitable system or device, or any combination thereof.

Step 1702 may include a patient monitoring system performing a wavelet transform (e.g., a continuous wavelet transform) on a time-domain physiological signal. In some embodiments, step 1702 may be performed across a range of scales. Step 1702 may output wavelet transform parameters including, for example, the transform values themselves (e.g., T(a,b) of equation (14)), energy distribution values (e.g., S_{R}(a,b) of equation (15)), relative phase difference values, any other suitable derived values, any suitable mathematical manipulations thereof (e.g., scaled S_{R}(a,b) of equation (16)), or any combination thereof. In some embodiments, step 1702 may include generating a scalogram.

Step 1704 may include a patient monitoring system performing a template match between the outputted wavelet transform parameters of step 1702 and one or more templates. Step 1704 may include performing a correlation calculation (e.g., calculating a covariance value for each comparison with each template), performing a difference calculation, performing any other suitable calculation between calculated wavelet transform parameters and one or more templates, or any combination thereof.

Step 1706 may include a patient monitoring system determining scale variability. Step 1706 may include monitoring a physiological signal, calculating one or more scale variability values at one or more scales, recalling a scale variability stored in memory, any other suitable determination, or any combination thereof.

Step 1708 may include a patient monitoring system combining the template matching results of step 1704 with the scale variability of step 1706. Step 1708 may include multiplying template matching results with scale variability (e.g., at each scale, at each scale using a weighting based on scale), summing template matching results with scale variability (e.g., at each scale, at each scale using a weighted sum with a weighting based on scale), scaling either or both template matching results and scale variability, any other suitable mathematical combination, or any combination thereof.

Step 1710 may include a patient monitoring system processing the combined signal of step 1708. Step 1710 may include averaging the combined signal (e.g., time averaging, ensemble averaging, weighted averaging), calculating a peak of the combined signal, filtering the combined signal, filtering a location of a peak value of the combined signal, averaging location of a peak value, computing one or more confidence metrics, any other suitable processing of the combined signal, or any combination thereof. For example, the combined signal of step 1708 may be low-pass filtered (over time) to limit rates of temporal change of the combined signal. In a further example, a recursive average, using an infinite impulse response (IIR) filter, may be calculated for the combined signal over time. In a further example, the location of the peak value may correspond to a BPM value. The BPM value, which may be outputted to a user, may be low-pass filtered (over time) to limit the rate of change in the outputted BPM value (e.g., to match an expected physiological range of heart rate).

In some embodiments, step 1710 may include a patient monitoring system outputting a combined signal, physiological parameter calculated thereof, any other suitable information, or any combination thereof. Outputting may include displaying, providing for further calculation, storing, any other suitable steps, or any combination thereof. For example, the patient monitoring system may display calculated physiological parameter values as a time series, an alphanumeric text box, any other suitable visual representation, or any combination thereof. In a further example, the patient monitoring system may display one or more calculated confidence metrics to a user to indicate signal quality, confidence in a physiological parameter determination, or both. In a further example, the patient monitoring system may provide information for performing further calculations, evaluations, or both (e.g., determine whether to activate alarms, or calculate additional metrics). In a further example, the patient monitoring system may store the information such as physiological parameter values, confidence metrics, combined signals, scale variability, any other suitable information, or any combination thereof, in memory.

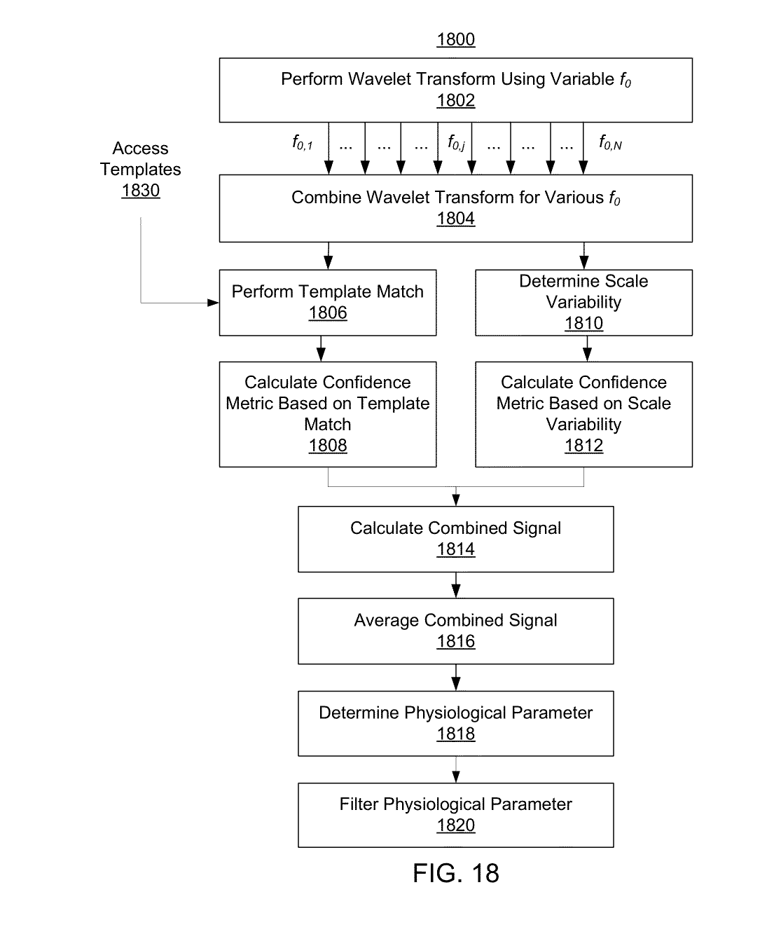

FIG. 18 is a flow diagram 1800 showing illustrative steps for determining a physiological parameter using template matching, scale variability, and confidence metrics, in accordance with some embodiments of the present disclosure. Any or all of the steps of flow diagram 1800 may be performed by a suitable patient monitoring system (e.g., patient monitoring system 10 of FIGS. 1 and 2), any other suitable system or device, or any combination thereof.

Step 1802 may include a patient monitoring system performing a wavelet transform on a time-domain physiological signal at a plurality of scales using wavelets having varying oscillatory character (e.g., f₀). In some embodiments, step 1802 may be performed across a range of scales. Step 1802 may output wavelet transform parameters including, for example, the transform values themselves (e.g., T(a,b) of equation (14)), energy distribution values (e.g., S_{R}(a,b) of equation (15)), relative phase difference values, any other suitable derived values, any suitable mathematical manipulations thereof (e.g., scaled S_{R}(a,b) of equation (16)), or any combination thereof. In some embodiments, step 1802 may include generating a scalogram.

Step 1804 may include a patient monitoring system combining the results of step 1802 for each f₀, outputting a wavelet transform, outputting any suitable parameters derived from a wavelet transform (e.g., an energy distribution, a relative phase difference), any other suitable steps, or any combination thereof.

Although shown illustratively in FIG. 18 as using wavelets having varying oscillatory character, a patient monitoring system may use any suitable wavelets, having any suitable characteristics, in accordance with the present disclosure. In some embodiments, step 1802 need not be performed using wavelets having varying oscillatory character. For example, a wavelet transform may be performed using a constant f₀ value, and accordingly the combining in step 1804 need not be performed.

Step 1806 may include a patient monitoring system performing a template match between the outputted wavelet transform parameters of step 1804 and one or more templates, accessed at step 1830. Step 1806 may include performing a correlation calculation (e.g., calculating a covariance value for each comparison with each template), performing a difference calculation, performing any other suitable calculation between calculated wavelet transform parameters and one or more templates, or any combination thereof.

Step 1808 may include a patient monitoring system calculating one or more confidence metrics based at least in part in the template matching results of step 1806. Confidence metrics may include a correlation value of the best “match” (i.e., the peak value), a covariance value between template matching results and the template corresponding to the best match to provide a confidence metric, any other suitable metric, or any combination thereof. Any suitable calculated value may be used as a confidence metric.

Step 1810 may include a patient monitoring system determining scale variability. Step 1810 may include monitoring a physiological signal, calculating one or more scale variability values at one or more scales, recalling a scale variability value stored in memory, any other suitable determination, or any combination thereof.

Step 1812 may include a patient monitoring system calculating one or more confidence metrics based at least in part on the scale variability of step 1810. Any suitable calculated value may be used as a confidence metric.

Step 1814 may include a patient monitoring system combining the template matching results of step 1806 with the scale variability of step 1810. Step 1814 may include multiplying template matching results with scale variability (e.g., at each scale, at each scale using a weighting based on scale), summing template matching results with scale variability (e.g., at each scale, at each scale using a weighted sum with a weighting based on scale), scaling either or both template matching results and scale variability (e.g., using confidence metrics of steps 1808, 1812, or both), any other suitable mathematical combination, or any combination thereof.

Step 1816 may include a patient monitoring system averaging the combined signal of step 1814. In some embodiments, confidence metrics, such as those calculated in either or both of steps 1808 and 1812, may be used to determine how the averaging is to be performed. For example, for relatively low confidences, averaging may be performed over relatively larger time intervals or number of samples. Step 1816 may include averaging the combined signal at each scale (e.g., a moving average in time for each relevant scale), using a scale-weighted average across scale, calculating statistical metrics of the averaged combined signal (e.g., standard deviation, expected values), any other suitable calculations, or any combination thereof. For example, a recursive average, using an infinite impulse response (IIR) filter, may be calculated for the combined signal over time.

Step 1818 may include a patient monitoring system determining a physiological parameter based at least in part on the averaged combined signal of step 1816. Step 1818 may include determining a location (e.g., scale) of a peak value of the combined signal, mathematically manipulating the peak value or location, using a look-up table based on the location, any other suitable determinations, or any combination thereof. In some embodiments, a pulse rate may be determined by computing the period of the real portion of a selected scale (e.g., the scale corresponding to the peak value of the combined signal or the template matching results) based on fiducial analysis (e.g., determining pulse period based on fiducial points of successive pulse waves). In some embodiments, especially when there is limited scale resolution (e.g., due to computational limitations of hardware, software, or both), the use of the selected scale and fiducial analysis may be advantageous.

Step 1820 may include a patient monitoring system filtering the determined physiological parameter of step 1818. Step 1820 may include low-pass filtering to limit the rate of change of the determined physiological parameter (e.g., to physiological ranges), limiting the determined physiological parameter to particular numerical ranges, smoothing the determined physiological parameter, any other suitable filtering, or any combination thereof. In some embodiment, the filtered physiological parameter values may be outputted by a patient monitoring system (e.g., for display, further calculation, or storage). For example, the patient monitoring system may display the physiological parameter values as a time series, an alphanumeric text box, any other suitable visual representation, or any combination thereof. In a further example, the patient monitoring system may perform further calculations, evaluations, or both, on the physiological parameters (e.g., determine whether to activate alarms such as “low pulse rate,” or calculate additional metrics). In a further example, the patient monitoring system may store the physiological parameter values in memory.

In some embodiments, one or more steps of flow diagram 1800 need not be performed. For example, in some embodiments, the averaging of step 1816 need not be performed, and the physiological parameter of step 1818 may be determined based on an un-averaged combined signal. In a further example, in some embodiments, the filtering of step 1820 need not be performed, and a patient monitoring system may output unfiltered physiological parameter values.

Any of the illustrative steps of flow diagrams 1600-1800 may be combined with other steps, omitted, rearranged, or otherwise altered in accordance with the present disclosure.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof which are within the spirit of the following claims.

What is claimed is:

1. A method for determining a physiological parameter, the method comprising:
calculating, using a processor, wavelet transform parameters for a physiological signal;
calculating, using a processor, a plurality of comparisons of the wavelet transform parameters with each of a plurality of templates;
selecting, using a processor, a comparison of the plurality of comparisons based at least in part on the plurality of comparisons; and
determining, using a processor, a value of the physiological parameter based at least in part on the selected comparison.

2. The method of claim 1, further comprising:
determining a scale variability signal based at least in part on the wavelet transform parameters; and
calculating, using a processor, a combined signal based at least in part on the plurality of comparisons and the scale variability signal.

3. The method of claim 2, further comprising repeating the steps of claim 2 at least once to produce two or more combined signals, and the method further comprising averaging the two or more combined signals.

4. The method of claim 2, wherein the plurality of templates comprises a plurality of energy distribution templates, and wherein the calculating the combined signal comprises combining the plurality of calculated comparisons with the scale variability signal by performing one or more of multiplying at each scale, multiplying at each scale using a weighted product, summing at each scale, and summing at each scale using a weighted sum.

5. The method of claim 2, wherein the determining the value of the physiological parameter is further based at least in part on a confidence metric derived from the scale variability signal.

6. The method of claim 1, wherein the determining the value of the physiological parameter is further based at least in part on a confidence metric derived from the plurality of comparisons.

7. The method of claim 1, wherein the calculating the wavelet transform parameters for the physiological signal comprises using a wavelet whose oscillatory character depends on a scale, wherein the oscillatory character changes by one or more of stepwise decreasing as scale increases, linearly decreasing as scale increases, and nonlinearly decreasing as scale increases.

8. The method of claim 1, wherein the plurality of templates comprises a plurality of relative phase difference distributions, and wherein the wavelet transform parameters comprise relative phase differences, and wherein the calculating the plurality of comparisons further comprises calculating a comparison of the relative phase differences of the wavelet transform parameters with each of the plurality of relative phase difference distributions.

9. The method of claim 1, wherein the calculating the plurality of comparisons comprises calculating a plurality of correlations between the wavelet transform parameters and each of the plurality of templates.

10. The method of claim 1, further comprising repeating the steps of claim 1 at least once to produce two or more values of the physiological parameter, the method further comprising applying a filter to the two or more values of the physiological parameter.

11. A system for determining a physiological parameter, the system comprising:
one or more sensors configured to detect a physiological signal;
memory configured to store a plurality of templates; and
one or more processors coupled to the memory and to the one or more sensor, the one or more processors configured to:
calculate wavelet transform parameters for the physiological signal;
calculate a plurality of comparisons of the wavelet transform parameters with each of the plurality of templates;
select a comparison of the plurality of comparisons based at least in part on the plurality of comparisons; and
determine a value of the physiological parameter based at least in part on the selected comparison.

12. The system of claim 11, wherein the one or more processors are further configured to:
determine a scale variability signal based at least in part on the wavelet transform parameters; and
calculate a combined signal based at least in part on the plurality of comparisons and the scale variability signal.

13. The system of claim 12, wherein the system is configured to:
calculate two or more combined signals; and
average the two or more combined signals.

14. The system of claim 12, wherein the plurality of templates comprises a plurality of energy distribution templates, and wherein the calculating the combined signal comprises combining the plurality of calculated comparisons with the scale variability signal by performing one or more of multiplying at each scale, multiplying at each scale using a weighted product, summing at each scale, and summing at each scale using a weighted sum.

15. The system of claim 12, wherein the one or more processors are further configured to determine the value of the physiological parameter further based at least in part on a confidence metric derived from the scale variability signal.

16. The system of claim 12, wherein the one or more processors are further configured to determine the value of the physiological parameter further based at least in part on a confidence metric derived from the plurality of comparisons.

17. The system of claim 11, wherein the one or more processors are further configured to calculate the wavelet transform parameters for the physiological signal using a wavelet whose oscillatory character depends on a scale, wherein the oscillatory character changes by one or more of stepwise increasing with the scale, linearly increasing with the scale, nonlinearly increasing with the scale, and a combination thereof.

18. The system of claim 11, wherein the plurality of templates comprises a plurality of relative phase difference distributions, and wherein the wavelet transform parameters comprise relative phase differences, and wherein the calculating the plurality of comparisons further comprises calculating a comparison of the relative phase differences of the wavelet transform with each of the plurality of relative phase difference distributions.

19. The system of claim 11, wherein one or more processors is further configured to calculate the plurality of comparisons using a plurality of correlations between the wavelet transform parameters and each of the plurality of templates.

20. The system of claim 11, wherein the system is configured to:
calculate two or more values of the physiological parameter; and
apply a filter to the two or more values of the physiological parameter.
